(19) United States
(12) Patent Application Publication
(10) Pub. No.: US 2022/0313848 A1
(43) Pub. Date: Oct. 6, 2022

(54) Mist Spray Device and Mist Shower Device for Sterilization and Disinfection using the Same

(71) Applicant: SOOVON Co., Ltd., Incheon (KR)

(72) Inventor: Kyoung Hee OH, Goyang-si (KR)

(21) Appl. No.: 17/224,084

(22) Filed: Apr. 6, 2021

(30) Foreign Application Priority Data
Apr. 1, 2021 (KR) 10-2021-0042656

(51) Int. Cl. A61L 2/00; A61L 2/26; B05B 17/06; B05B 3/02

(52) U.S. Cl. CPC A61L 2/0088; A61L 2/26; B05B 17/06; B05B 3/022

(57) ABSTRACT

The mist spray device according to the present invention is formed by including a mist generator configured to generate mists to above water surface of the water contained in a water tank, an air blower and an air guide part configured to form an air passage to allow the air flow generated by the air blower to be moved to a mist discharge port. Particularly, the water tank is disposed with an inlet through which part of the air flow generated by the air blower is introduced and an outlet through which the mists generated by the mist generator are discharged to the air passage, and an amount of air introduced into the water tank can be adjusted using the air inflow adjustment part.

### CROSS-REFERENCE TO RELATED APPLICATION

This application claims priority to and the benefit of Korean Patent Application No. 10-2021-0042656 filed on Apr. 1, 2021 in the Korean Intellectual Property Office, the entire content of which is incorporated herein by reference.

### TECHNICAL FIELD

The teachings in accordance with exemplary and non-limiting embodiments of this invention relate generally to a mist spray device configured to spray mist along with air by generating the mist, and a mist shower device for sterilization and disinfection configured to perform sterilization and disinfection by spraying, by way of mist, sterilization water generated through electrolysis, using the said mist spray device.

### BACKGROUND OF THE INVENTION

Recently, corona-19 virus outbreak around the world has a huge impact on everyday life and an efficient quarantine measures are more urgent than anything else against a large number of people entering and leaving various places in everyday life.

Unlike the livestock epidemics, people are infected with the corona-19 throughout the regions, and quarantines are required against visitors in most of places where people enter and leave.

In relation therewith, a disinfecting (sterilizing) room traditionally installed in limited places such as cattle sheds for performing a full body disinfection and sterilization against entrants is not easy to install, cumbersome to use, and therefore, difficult to use efficiently in many places.

Furthermore, because many visitors cannot wear their own quarantine suits one by one, sterilization and disinfection must be performed while being dressed in everyday clothes, and there should be no side effects in this process that cause harm to the human body or contaminate clothing, depending on compositions or conditions of substances used in the sterilization and disinfection.

In relation to sterilization and disinfection devices against entrants, the Korean registered patent No.: 10-2191093 entitled ‘Outdoor Walking Through Disinfecting Device’ discloses that the whole body of a passer-through is made to be sterilized against viruses while being installed outdoors and an atmosphere of disinfectant (water) is created as a person passes therethrough.

However, the traditional sterilization and disinfection devices like the said Korean registered patent No.: 10-2191093 use disinfectants that are mostly manufactured separately and distributed by being put in individual containers.

For example, the disinfectants used in the said Korean registered patent No.: 10-2191093 are also distributed while stored in the disinfection water storage (container) and therefore contents therein must be periodically refilled or otherwise, the whole contents must be refilled for use.

When disinfectant water distributed while being contained in individual containers are used as explained above, the supply and management of disinfection water are very inconvenient. Periodic and regular supply of disinfectants into sterilizing and disinfecting devices installed in multiple locations require a considerable amount of time and manpower, and therefore are hardly cost-effective.

Electrolysis method may be used in relation to sterilization water generation technology useable for sterilization and disinfection.

For example, the Korean registered patent No.: 10-2161372 discloses the ‘Portable Beauty Hydrogen Sterilization Mist Device’ that generates sterilization water by discharging raw water underwater and discharges outside the generated sterilization water in a mist type.

However, the conventional technology for generation of sterilization water using electrolysis such as the Korean registered patent No.: 10-2161372 is largely used for generation of small-scale sterilization water as in beauty purposes, and not briskly used in such fields that require a large amount of sterilization water as in the quarantines against a large number of people. The electrolysis method can supply sterilization water conveniently and economically, and may be adequately utilized under situations where quarantine is carried out in daily life due to corona-19 pandemic and the like.

Meantime, it is necessary to spray mists of fine particles for diverse purposes such as spraying the mists to persons subject to quarantine for sterilization and disinfection.

For example, the said Korean registered patent No.: 10-2191093 provides pressure to allow, using a fan, the disinfectant water in a sprayed condition to be transferred.

However, various factors are considered to spray a large quantity of mists using a fan, including, but not limited to, the number of fans, numerous arrangements of fans, weight of device, mist generation properties by mist generation method, mist spray efficiency and the like.

Particularly, when mists are generated using an ultrasonic oscillator, mists must be sprayed under an adequate pressure and quantity lest the mists be extinguished.

### RELATED TECHNICAL DOCUMENTS

### Patent Documents

(1) Korean registered patent No.: 10-2191093 (Title: Outdoor Walking Through Disinfecting Device, published on Dec. 15, 2020)
(2) Korean registered patent No.: 10-2161372 (Title: Portable Beauty Hydrogen Sterilization Mist Device, published on Jul. 17, 2020)

### SUMMARY OF THE INVENTION

### Technical Subject

The present invention is devised to meet the requirements of the aforementioned disadvantages/problems and it is an object of the present invention to provide a mist spray device configured to efficiently spray mists using a minimum number of fans by adequately distributing the flow of generated air.

It is another object of the present invention to provide a mist shower device for sterilization and disinfection configured to supply convenient and economic disinfectant water using electrolysis and to allow sterilizing mists to be efficiently sprayed.

### Technical Solution

In one general aspect of the present invention, there may be provided a mist spray device, the device comprising:

a water tank configured to contain water that is to be transformed to mists;

a mist generator configured to generate mists to above water surface of the water contained in the water tank;

an air blower configured to generate an air flow; and

an air guide part configured to form an air passage to allow the air flow generated by the air blower to be moved to a mist discharge port.

At this time, the water tank may be disposed with an inlet through which part of the air flow generated by the air blower is introduced and an outlet through which the mists generated by the mist generator are discharged to the air passage.

Preferably but not necessarily, the inlet and outlet may be mutually oppositely disposed above an upper surface of the air tank.

At this time, the mist spray device may further comprise a mist inducement means to allow the air flow introduced into through the inlet to be induced to one side of the water tank.

Preferably but not necessarily, the mist inducement means may be formed with a shape of a plate descending at a predetermined angle from a side formed with the outlet toward a descending side formed with the inlet.

The mist spray device according to the present invention may further comprise an air inflow adjustment part configured to adjust an amount of air introduced into the water tank through the inlet.

Preferably but not necessarily, the mist spray device according to the present invention may further comprise:

one or more electrolysis modules configured to supply sterilization water to the water tank by generating the sterilization water having a sterilizing power through electrolysis; and

one or more porous members disposed inside of the water tank to allow mists heading for the outlet to pass therethrough and to drop the mists by condensing thick mist particles.

Preferably but not necessarily, the mist generator may be formed by including one or more ultrasonic oscillators disposed underneath the water tank.

Preferably but not necessarily, the air guide part may be formed with a shape to encompass a surface formed with the inlet of the water tank, an upper surface of the water tank and a surface formed with the outlet of the water tank, and a corner of the water tank may be formed with a slope at a portion where the direction of air flow is changed.

Furthermore, at least one or more air passages may be formed by being narrowed to facilitate the air flow.

In another general aspect of the present invention, there may be provided a mist shower device for sterilization and disinfection, the device comprising: a housing disposed to form a passage to allow a person subject to quarantine to pass therethrough; and the said mist spray device described above.

Preferably but not necessarily, the housing may include an upper side housing forming a ceiling, a left side housing forming a left side wall and a right-side housing forming a right-side wall.

At this time, the mist spray device may be disposed on an upper side housing.

Furthermore, the mist shower device for sterilization and disinfection may be so disposed as to spray sterilizing mists downwardly from the upper side housing.

### Advantageous Effects

The mist spray device according to the present invention can discharge mists generated from an upper space above a water surface of water tank to an outlet by inducing and supplying some of the air flows generated from an air blower into an interior of the water tank.

There is no need to separately use an air blower for generating air flow and an air blower for discharging mists to thereby enable an efficient spray of mists through a minimum number of fans.

Concomitant with the use of a minimum number of air blowers, both cost reduction may be achieved and weight of device can be reduced to thereby enable a convenience maintenance.

The air flow introduced into the water tank can be smoothly induced above a water surface of the water tank through a mist inducement means to enable a smooth discharge of mists generated by an ultrasonic oscillator without being extinguished.

Furthermore, the mist shower device for sterilization and disinfection according to the present invention can directly generate sterilizing water using electrolysis to dispense with the cumbersome need of repeated supply of sterilizing water (disinfecting water) in an individual container whereby economic, convenient, easy, and comfortable maintenance can be greatly achieved.

Because the size of mist particles is very fine and the mist particles are filtered once again through porous members, water drops are prevented from falling and clothes of a person subject to quarantine are avoided from being wetted, whereby safety accidents caused by slip can be prevented thanks to a floor not being wetted.

The sterilizing mists can be sprayed downwardly from above a head of a person subject to quarantine to thereby enhance a full body sterilizing effect.

### BRIEF DESCRIPTION OF DRAWINGS

FIG. 1 is a mist spray device according to an exemplary embodiment of the present invention.

FIGS. 2, 3 and 4 are a cross-sectional view of mist spray device according to a detailed exemplary embodiment of the present invention, a view seen from above while an air guide part is removed according to a detailed exemplary embodiment of the present invention, and a view seen from a lateral surface according to a detailed exemplary embodiment of the present invention.

FIG. 5 is an electrolysis module according to an exemplary embodiment of the present invention.

FIG. 6 is an air inflow adjustment part according to an exemplary embodiment of the present invention.

FIG. 7 is a mist shower device for sterilization and disinfection according to an exemplary embodiment of the present invention.

FIG. 8 explains an example where a portion contacted by mists is changed to generate a drying in response to movement of mesh plate.

FIG. 9 is a mesh plate driving part according to an exemplary embodiment of the present invention.

FIG. 10 is an enlarged view of a mesh plate driving part according to an exemplary embodiment of the present invention.

FIG. 11 explains an example where mist spray conditions (states) are adjusted when a spray condition adjustment part ascends/descends relative to a mist discharge port.

FIGS. 12 and 13 illustrate a spray condition (state) adjustment part according to a detailed exemplary embodiment of the present invention.

### DETAILED DESCRIPTION

The present invention may be applied with various changes and have several exemplary embodiments, where particular exemplary embodiments will be exemplified in the drawings and described in detail through the detailed description of the present invention.

However, it should be understood that the present invention is not limited to particular embodiments, but encompasses all changes, modifications, equivalents and substitutes included within the ideas and technical scopes of the present invention.

In describing the present invention, detailed descriptions of well-known technologies are omitted for brevity and clarity so as not to obscure the description of the present invention with unnecessary detail.

The terminology used herein is for the purpose of describing exemplary embodiments only and is not intended to be limiting. As used herein, the singular forms may be intended to include the plural forms as well, unless the context clearly indicates otherwise.

It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be understood that, although the terms first, second, etc. may be used herein to describe various elements, these elements should not be limited by these terms. These terms are only used to distinguish one element from another.

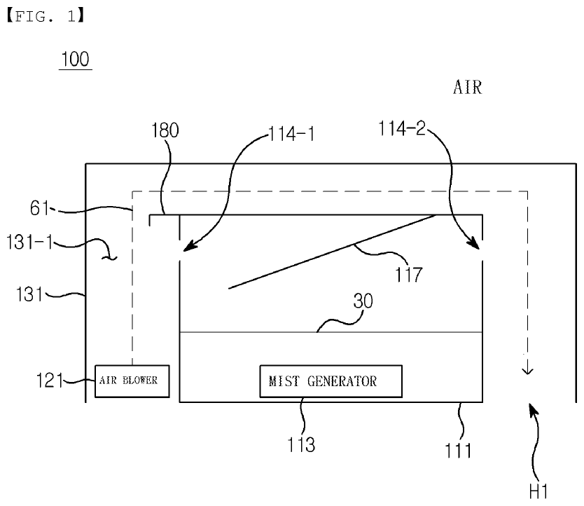

FIG. 1 is a mist spray device 100 according to an exemplary embodiment of the present invention, the device 100 comprising: a water tank configured to contain water that is to be transformed to mists; a mist generator 113 configured to generate mists to above water surface of the water contained in the water tank 111; an air blower 121 configured to generate an air flow 61; and an air guide part 131 configured to form an air passage 131-1 to allow the air flow 61 generated by the air blower 121 to be moved to a mist discharge port (H1).

At this time, the water tank 111 may be disposed with an inlet 114-1 through which part of the air flow generated by the air blower 121 is introduced and an outlet 114-2 through which the mists generated by the mist generator are discharged to the air passage 131-1.

The air flow generated by the air blower 121 may fly or flow toward the mist discharge port (H1) along the air passage 131-1, and part of the air flow may be introduced into an interior of the water tank 111 through the inlet 114-1.

The air flow introduced into the interior of the water tank 111 through the inlet 114-1 may move the mists generated within the water tank 111 and induce the mists to slip out to the air passage 131-1 through the outlet 114-2.

Furthermore, the mists slipped out to the air passage 131-1 through the outlet 114-2 of the water tank 111 may be sprayed to outside through the mist discharge port (H1) along with the air flow that flows through the air passage 131-1.

The water tank 111 may perform a role of a container containing water to be transformed to mists, and material, shape and size of the water tank 111 may be variably configured.

The mist generator 113 may generate mists to above the water surface 30 of the water contained in the water tank 111 and may be variably configured to transform the water contained in the water tank 111 to mists which are fine particles.

As one example, the mist generator 113 may be formed by including one or more ultrasonic oscillators disposed underneath the water tank 111. The ultrasonic oscillator may generate mists to above the water surface 30 of the water contained in the water tank 111.

The depth of water submerged by the ultrasonic oscillator may be maintained at a predetermined level to create an optimal environment capable of generating mists upon an upper space of the water surface 30.

The water tank 111 may be partitioned into a plurality of sections along a lengthwise direction, and each partitioned section may be disposed with one or more ultrasonic oscillators. As a result, mists can be more uniformly generated and discharged.

The air blower 121 may generate an air flow. The air blower 121 may be formed using various blowing technologies including, but not limited to, various types of fans and blowers to spray mists at a desired level.

Although the air blower 121 may be configured using factors including, but not limited to, one or more fans or blowers, the number of fans or blowers forming the air blower 121 can be minimized because use is made in the present invention by distributing the air flow generated from the air blower 121.

The air blower 121 may be disposed on various positions using various methods to generate the air flow and to allow the air flow to adequately flow.

The air guide part 131 may form an air passage 131-1 to allow the air flow generated by the air blower 121 to be flown toward the mist discharge port (H1).

The air passage 131-1 formed by the air guide part 131 may be variably formed as necessary. Although the figure shows an example of forming the air guide part 131 encompassing both lateral surfaces and an upper surface of water tank 111 along a lengthwise direction, the present invention is not limited thereto.

The mist discharge port (H1), through which the air flow 61 flowing along the air passage 131-1 is discharged along with the mists, may be variably formed.

The mists and air flow may be variably discharged depending on shapes, methods, and the sizes of the mist discharge port (H1).

As an example, when the mist is sprayed in a narrow and long shape, the mist discharge port (H1) may be formed with a long hole shape.

The water tank 111 may be disposed with an inlet 114-1 through which part of the air flow 61 generated by the air blower 121 is introduced, and an outlet 114-2 through which mists generated above the water surface 30 of the water tank are slipped out through to the air passage 131-1.

The locations, shapes, and structures of the inlet 114-1 and the outlet 114-2 may be variably formed. As a detailed example, the inlet 114-1 and the outlet 114-2 may be mutually oppositely formed above the water tank 111.

With reference to smooth discharge of mists formed above a water surface 30 of water tank, a mist inducement means 117 may be disposed at an interior of water tank 111 that induces the air flow introduced through the inlet 114-1 toward one side of the water tank 111.

The mist inducement means 117 may be variably formed. As an example, the mist inducement means 117 may be downwardly formed with a descending plate shape at a predetermined angle from a side formed with the outlet 114-2 toward a side formed with the inlet 114-1.

Then, the air flow (wind) introduced into an interior of the water tank 111 through the inlet 114-1 may return to a downward side formed with the inlet 114-1 through the mist inducement means 117. Furthermore, the air flow may flow above the water surface 30 to proceed toward the outlet 114-2 that is disposed at an opposite side, whereby the mists may be moved and smoothly discharged to the air passage 131-1 through the outlet 114-2.

When the wind direction within the water tank is processed with the method thus mentioned, the mist discharge efficiency using the air blower 121 can be increased.

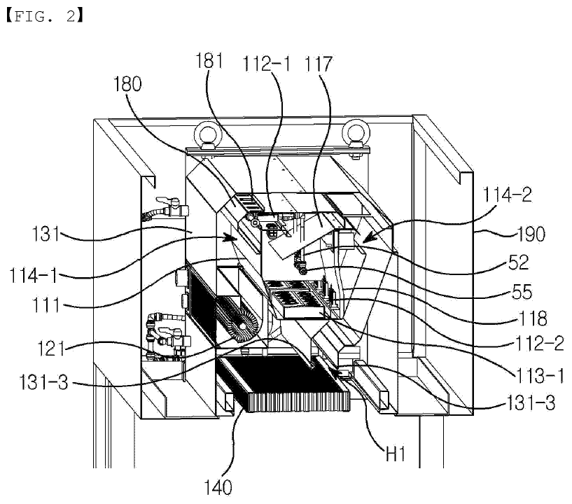

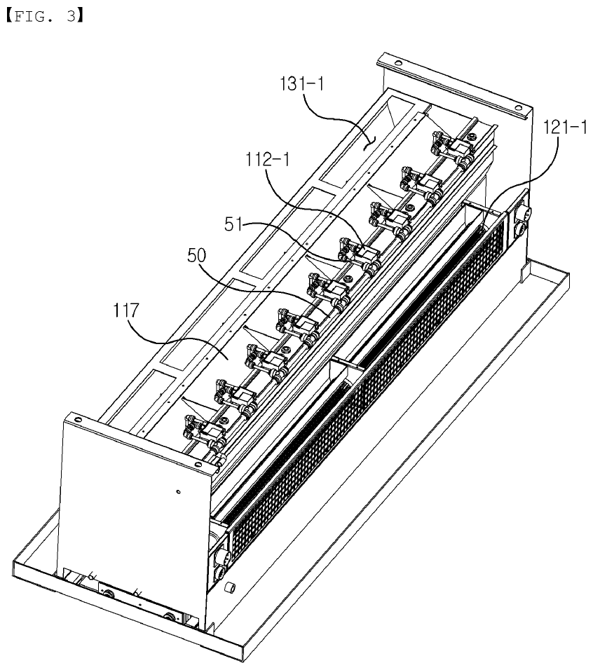

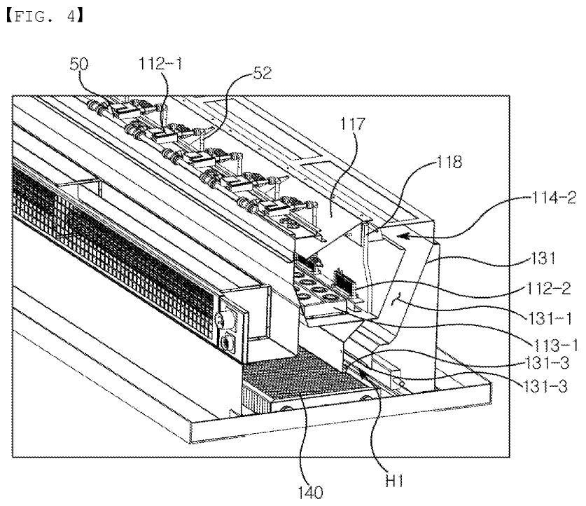

FIGS. 2, 3 and 4 are detailed views of mist spray device according to an exemplary embodiment of the present invention, where FIG. 2 is a cross-sectional view, FIG. 3 is an example seen from above while an air guide part is removed, and FIG. 4 is an exemplary view seen from a lateral surface. Some parts are omitted from the drawings in order not to distract the gist of the description.

The figure illustrates a shape in which a water tank 111 is lengthily formed inside a case 190 forming an external appearance of the mist spray device 100, and the air guide part 131 encompasses both lateral surfaces and an upper surface of water tank 111 toward a lengthwise direction.

The mist spray device 100 may be used in various fields that require the mist spray. As a detailed example, the mist spray device 100 may be used in a sterilization/disinfection fields where sterilization water is made for spray.

At this time, the mist spray device 100 may be formed by including one or more electrolysis modules 112-1, 112-2 configured to generate sterilization water having the sterilizing power (hereinafter referred to simply or sometimes as ‘sterilizable water’) using electrolysis and to supply the generated sterilization water to the water tank 111.

The electrolysis modules 112-1, 112-2 can generate the sterilization water with the sterilizing power by electrolyzing a raw water. The raw water may be supplied through various routes. For example, the electrolysis module may be so configured as to electrolyze the water contained in the water tank 111, or may electrolyze the raw water such as faucet water introduced through a raw water supply pipe 50 from an outside of the water tank 111 and supply the electrolyzed water to the water tank 111.

The electrolysis module may be disposed in various methods.

As an example, the electrolysis module 112-2 may electrolyze the water contained in the water tank 111 by being disposed underneath the water tank 111 to be submerged in the water contained in the water tank 111.

In another example of configuration, the electrolysis module 112-1 may be disposed at an upper surface of the water tank 111 so as not to be submerged in the water contained in the water tank 111, generate the sterilization water through the electrolysis and supply the generated sterilization water to the water tank 111.

In case of the configuration explained in the latter example, the electrolysis module 112-2 may not be submerged in the water to provide an advantage that there is less defect rate and it is convenient to repair and maintain the electrolysis module.

Although two examples 112-1, 112-2 are illustrated to explain some of various disposition methods of electrolysis modules, only any one of the disposition methods may be used.

The water tank 111 may be partitioned to several sections using diaphragms and a floor of each section may be disposed with a plurality of ultrasonic oscillators 113-1.

One or more electrolysis modules may be disposed to correspond to each section, and the raw water supplied along the raw water supply pipe 50 may be bifurcated to the raw water inflow pipe 51 and finally supplied to each electrolysis module 112-1.

Each electrolysis module 112-1 may generate the sterilization water by electrolyzing the supplied raw water and supply the generated sterilization water to the water tank through the sterilization water outlet pipe 52. A distal end of the sterilization water outlet pipe 52 may be bifurcated 55 to discharge the sterilization water to more than two areas above the water surface of relevant partitioned section.

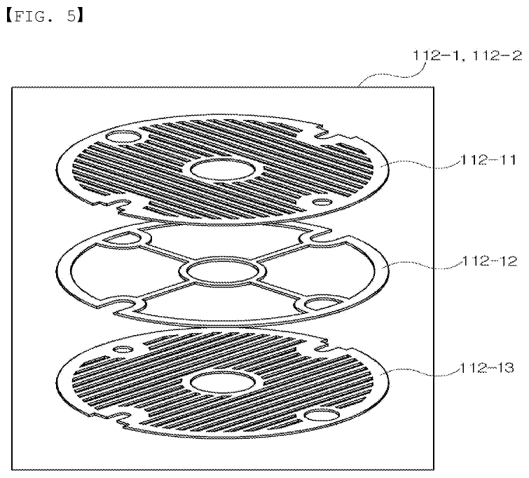

FIG. 5 illustrates the electrolysis modules 112-1, 112-2 according to an exemplary embodiment of the present invention, where the electrolysis module may be formed by including a pair or more pairs of electrode plates 112-11, 112-13 having a negative pole (−) and a positive pole (+), may disintegrate the water molecules using the underwater discharge principle and may generate the sterilization water having the sterilizing power.

For example, various negative ions (O⁻, O₃⁻, OH⁻, HOCl, H₂O₂) generated as a result of underwater plasma discharge through two electrode plates 112-11, 112-13 can sterilize various viruses like corona-19, germs, bacteria, and the like.

The electrolysis modules 112-1, 112-2 may be formed by including mutually opposing two electrode plates 112-11, 112-13 and an electrode separation plate 112-12 that allows the two electrode plates 112-11, 112-13 to be mutually spaced apart.

Each electrode plate 112-11, 112-13 may be formed with a plurality of slits to a particular direction, and the slit formed on each electrode plate may be so disposed as to mutually have a predetermined angle (e.g., 90 degrees) against the other slit. The water introduced into the electrolysis modules 112-1, 112-2 may be underwater discharged while passing through each slit.

FIG. 5 is provided as an example to help understand the overall explanation, and the electrolysis modules 112-1, 112-2 may be variably formed as necessary and the present invention is not limited thereto.

One or more electrolysis modules 112-1, 112-2, in the exemplary embodiment where the water tank 111 is partitioned to a plurality of sections, may be disposed against each section, where the electrolysis module and the ultrasonic oscillator 113-1 may be interacted on a one-on-one base, and also on a one-on-multiple number base as well.

The mist spray device 100 may be comprised by further including an air inflow adjustment part 180 configured to adjust an amount of airs introduced into the water tank 111 through the inlet 114-1, and a porous member 118 configured to allow mists toward the outlet 114-2 to pass therethrough.

The air inflow adjustment part 180 may be variably formed in order to adjust the amount of airs introduced into the water tank 111 through the inlet 114-1.

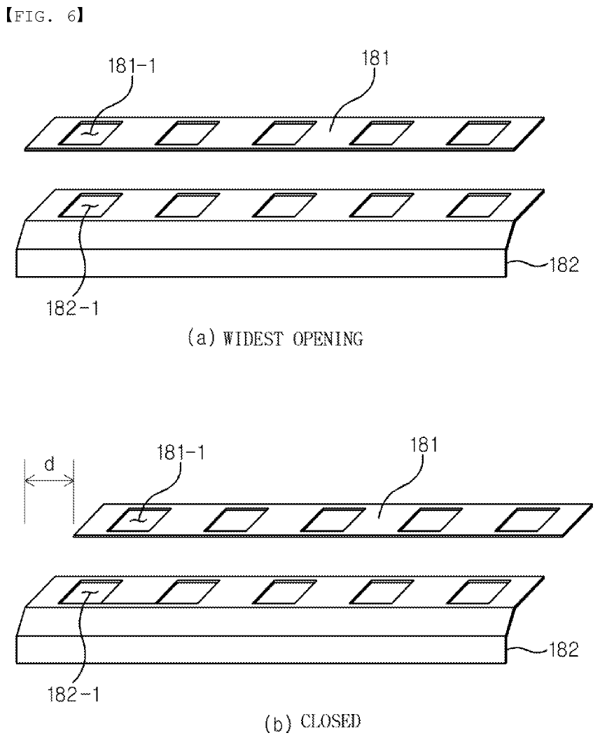

As a detailed example by referring to FIGS. 2 and 6, the air inflow adjustment part 180 may be disposed at an area, where air flow generated from the air blower 121 passes the inlet 114-1 formed on the water tank 111, to induce the air flow to proceed toward an inlet 114-1 direction by blocking some of the air flows.

The air inflow adjustment part 180 may include a main body part 182 formed with one or more air holes 182-1 configured to allow the air flow to pass therethrough and to induce the air flow to proceed toward an inlet 114-1 direction by blocking some of the air flows generated from the air blower 121, and an open/close adjustment part 181 configured to adjust the degree of openness of the air hole 182-1 formed on the main body part 182.

The open/close adjustment part 181 may be formed with a shape of plate formed with open/close hole 181-1 in response to each air hole 182-1 of the main body part 182. As a result, an area, where the air hole 182-1 and the open/close hole 181-1 are overlapped, may be changed depending on the location of the open/close adjustment part 181 to thereby adjust the degree of the air flow that passes.

As illustrated in FIG. 6a, when the air hole 182-1 and the open/close hole 181-1 mutually correspond in positions thereof, a largest amount of air can pass therethrough, and as a result a least amount of air may be introduced into the inlet 114-1. However, as illustrated in FIG. 6b, when the air hole 182-1 and the open/close hole 181-1 are completely out of positions thereof, the air flow is blocked to allow a largest amount of air to be introduced into the inlet 114-1.

When the open/close adjustment part 181 is out of position as much as ‘d’ while the air hole 182-1 is completely blocked, the amount of passing air may be determined by adjustment of position of the open/close adjustment part 181 that is made out of position (mismatched) as much as any value less than ‘d’, whereby an amount of air introduced into the inlet 114-1 formed on the water tank 111 can be determined.

Methods to fasten the open/close adjustment part 181 to a particular position may be variably implemented.

For example, the open/close adjustment part 181 may be fixed to a necessary location using screws and/or the like, and may be moved to other locations by an automated method using a motor.

The water tank 111 may be disposed therein with a porous member 118 configured to allow mists toward the outlet 114-2 to pass therethrough.

The porous member 118 may perform the role of allowing coarse particles in the mists slipping out to the air passage 131-12 through the outlet 114-2 to drop on the water surface by being condensed, and may be formed with various materials including sponges.

The porous member 118 may prevent the situation where the coarse particles slip out through the air passage 131-1 and are discharged to an outside.

The air guide part 131 may be variably configured to form the air passage 131-1. As one example, the air guide part 131 may be formed with a shape to encompass a surface formed with the inlet 114-1 of the water tank 111, an upper surface of the water tank 111 and a surface formed with the outlet 114-2 of the water tank 111 along a lengthwise direction.

At this time, a fan formed by the air blower 121 may be disposed underneath a surface formed with the inlet 114-1 of water tank 111. In the given exemplary embodiment, the air flow generated by the air blower 121 may rise along a lateral surface of the water tank 111 formed with the inlet 114-1, may be changed in direction and may be moved along an upper surface of the water tank 111.

Furthermore, the air flow may change the direction to a downward direction again, descend along a lateral surface of water tank 111 formed with the outlet 114-2, and may be discharged to an outside through the mist discharge port (H1).

In this process, some of the air flows may be introduced into the water tank 111 by the air inflow adjustment part 180 at a portion formed with the inlet 114-1, and meet the mist slipped out through the air passage 131-1 at a portion formed with the outlet 114-2.

To further facilitate the air flow, a corner of the water tank 111 is formed with a slope at a portion where the direction of air flow generated by the air blower 121 is curbed while being ascended, and a portion where the air flow flowing along an upper surface of the water tank 111 is downwardly curbed again.

Furthermore, at least one or more of the air passages 131-1 may be so formed as to be narrowed to further facilitate the air flow according to the Bernoulli's principle.

For example, a portion of upper lateral sloped area of the water tank 111 formed with the outlet 114-2 may be formed to become narrowed.

A mesh plate 140 formed with a plurality of holes may be disposed at a location adjacent to the mist outlet (H1) to allow the air flows and the mists to pass therethrough.

At this time, the mists discharged through the mist discharge port (H1) and the air flow may be sprayed to outside by passing through the mesh plate 140. The plurality of holes formed on the mesh plate 140 may provide linearity (straightness) to the mists and the air flows.

The shape, depth, size, and disposition of holes formed on the mesh plate 140 may be variably implemented. For example, the mesh plate 140 may be formed with a hexagonal shape, but the present invention is not limited thereto.

Referring to FIGS. 2 and 4, a distal end of air guide part 131 formed with the mist discharge port (H1) may be formed with a condensate collection groove 131-3 to allow water drops producible from condensed mists to collect therein while flowing down.

That is, even if the mist is formed with microscopic particles, water drops may be generated by causing the mist to condense at an area continuously contacted by the mist.

Therefore, when a condensate collection groove 131-3 is formed at the distal end of the air guide part 131, the problem of the mist-condensed water drops being flown down and dropped can be prevented.

Although the figures have illustrated an example where distal ends of air guide part 131 are respectively folded inwardly to form the condensate collection grooves 131-3, the condensate collection groove 131-3 is not limited thereto and may be formed by various other methods.

Furthermore, albeit not separately illustrated, the outlet 114-2 of water tank 114-2 may be so configured as to be opened and closed. At this time, the mist spray device 100 may be used for the purpose of air shower curtain that uses only wind while the outlet 114-2 of water tank is closed.

Referring to FIG. 7, a mist shower device 200 for sterilization and disinfection (hereinafter referred to simply as ‘mist shower device’) according to the present invention may perform the sterilization and disinfection to persons subject to quarantine (hereinafter referred to simply as ‘quarantinable persons’) by generating sterilization water through electrolysis of raw water and spraying the generated sterilization water in the shape of mists.

The mist shower device 200 may be formed by including a housing forming a route 80 to be passed by the quarantinable persons, and a mist spray device 100 disposed on the housing to spray the mists having the sterilizing power.

The housing 200 of the mist shower device may be formed by including an upper side housing 210 forming a ceiling, a left side housing 220 forming a left side wall and a right-side housing 230 forming a right-side wall, whereby the route 80 to be passed by the quarantinable persons can be formed.

At this time, the mist spray device 100 may be disposed on an upper side housing 210, and the mist discharge port (H1) may be so disposed as to spray sterilizing mists downwardly from the upper side housing 210.

In consideration of the properties of mists generated from sterilization water in microscopic particles, it may be preferable that spraying be made from a ceiling toward a floor to increase the sterilization and disinfection effect to a full body of a quarantinable person instead of spraying from a lateral surface.

The water tank 111 of the mist spray device 100 may be contained with sterilization water for generation of sterilizable mists, and the sterilization water contained in the water tank 111 may be provided through various routes.

As an example, the electrolysis module 112-1 disposed above the water surface of the water tank 111 may be so configured as to generate sterilization water by electrolyzing the water contained in the water tank 111 or the raw water supplied from outside and to supply the sterilization water.

In another example, the electrolysis module 112-2 so disposed as to be submerged underneath the water tank 111 may electrolyze the water in the water tank 111 and generate the sterilization water.

In still another example of configuration, sterilizable water may be generated using a water tank provided separately from the mist spray device 100 and the electrolysis modules, and the generated sterilization water may be supplied to the water tank of the mist spray device 100.

At this time, the separate water tank and the electrolysis module may be disposed at any one or more of the left side housing 220 and the right side housing 230. In the given exemplary embodiment, the mist spray device 100 disposed on the upper side housing 210 may not be provided with a separate electrolysis module, and may also generate the mists using only the sterilization water supplied from outside.

In addition, the mist shower device 200 may be so disposed as to implement a repetitive electrolysis of more than twice.

For example, the sterilization water may be generated using the water tanks separately provided to the left side housing 220 and the right-side housing 230 and the electrolysis, and the said generated sterilization water may be again secondarily electrolyzed through an electrolysis module mounted on the mist spray module 100.

The mist shower device 200 may be disposed with a means for movement of sterilization water such as a pipe, a pump (not shown) and the like.

The housing may be disposed with a control panel 251 where various operational controls and displays for mist shower device 200 are implemented including power ON/OFF and operational condition displays, and a transparent window 253 may be also disposed to allow a user to visually and interestingly ascertain the process of generating the sterilization water.

That is, as shown in the figure, while the left side housing 220 is disposed with a water tank and an electrolysis module, the left side housing 220 may be disposed with a transparent window 253 that visually shows a process of the sterilization water being generated. At this time, the water tank interconnected with the transparent window 253 may be partially formed at least with a transparent material.

Furthermore, the mist shower device 200 may be so disposed as to adequately adjust the generation and spray of sterilization water according to various conditions including access/non-access of quarantinable subjects/persons and sterilization water generation condition (amount of sterilization water to be transformed to mists).

As an example, when an amount of sterilization water stored in the water tank is less than a predetermined level, the mist shower device 200 may be so operated as to control the implementation of electrolysis. Toward this end, one or more sensors (not shown) may be disposed to detect the level in the water tank, and the mist shower device 200 may be operated according to the condition detected through a relevant sensor.

In still another example, the mist shower device 200 may include a sensor (not shown) detectable of access of a quarantinable subject or person, and when the quarantinable subject or person was detected by the sensor, the mist shower device 200 may generate and spray the mists.

Now, a mist spray device 100 according to still another exemplary embodiment of the present invention will be described.

<Arrangement of Electrolysis Module>

The water tank 111 may be partitioned to a plurality of sections, and each partitioned section may be disposed with one or more ultrasonic oscillators 113-1. As a result, mists can be more uniformly generated and discharged. At this time, one or more electrolysis modules 112-1 may be disposed to correspond to each section.

When the ultrasonic oscillator 113-1 can best generate the mists from the water surface above a vertical direction thereof, it may be preferable that the sterilization water generated from the electrolysis module 112-1 through the electrolysis be supplied to a vicinity of the water surface above the vertical direction of an area disposed with each ultrasonic oscillator 113-1. It is because the sterilization water having the strongest sterilizing power has the possibility of being transformed to mists.

That is, the position of ultrasonic oscillator 113-1 and the sterilization water discharging position of electrolysis module 112-1 may affect each other with reference to the sterilizing power of mists.

In relation thereto, the electrolysis module 112-1 may be disposed above the water tank 111 in order not to be submerged into the water contained in the water tank 111, whereby the electrolysis module 112-1 can generate the sterilization water through electrolysis and supply the generated sterilization water to the water tank 111. At this time, the sterilization water generated from the electrolysis module 112-1 through the electrolysis may be so disposed as to be supplied to a vicinity above the water surface of an area disposed with the ultrasonic oscillator 113-1.

As a result, the sterilizing power of mists can be maximized. Furthermore, the defect rate can be reduced, and repair/maintenance can be more conveniently implemented because the electrolysis module 112-1 is not submerged in the water of the water tank.

<Prevention of Fall of Waterdrops>

Special attention and care are required in handling moisture caused by condensation of mists when the mists are sprayed.

When waterdrops fall due to condensation of mists, clothes may be wetted to cause inconveniences, passages may become slippery to result in generation of safety accidents, and the aesthetic environment may be aggravated to thereby cause lots of problems and disadvantages.

The mesh plate 140 is an element through which mists are discharged by passing therethrough and continuously contacts the mists as the mist spray device 100 is driven, such that even if mists are formed with microscopic molecules, waterdrops may be condensed due to continued contact with the mists.

One of the methods to dry the mesh plate 140 is that the outlet 114-2 of water tank is configured to be in an openable and closeable manner, and when drying is required, the outlet 114-2 of water tank configured to allow the mists to slip out to the air passage 131-1 is made to close and the mist spray device 100 is made to drive in an air shower mode that uses wind only.

Then, because only the air flow (wind) without the mists passes the mesh plate 140, the mesh plate 140 can be quickly dried.

Another method for drying the mesh plate 140 is that the mesh plate 140 is made to move an area where the air flow and the mists are contacted.

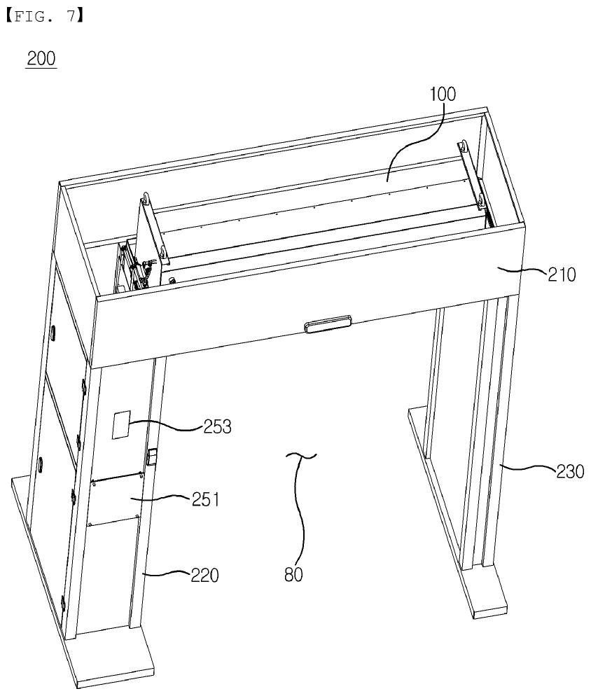

Referring to FIG. 8a, mists are currently discharged through a portion (M1) of the mesh plate 140, and the portion (M1) may capture the waterdrops due to continued spray of mists, and resultantly the waterdrops may fall to the floor.

Therefore, as shown in an example of FIG. 8b, an area contacted by the mists is replaced by another portion (M2) of mesh plate 140, whereby drying can be implemented before mists are condensed on a particular area of mesh plate 140.

At this time, it may be preferable that the width of mesh plate 140 be at least twice greater than that of the mesh plate discharge port (H1).

In this exemplary embodiment, the mist spray device 100 may be configured by including a mesh plate driving part 150 that changes an area contacted by the mists by driving the mesh plate 140.

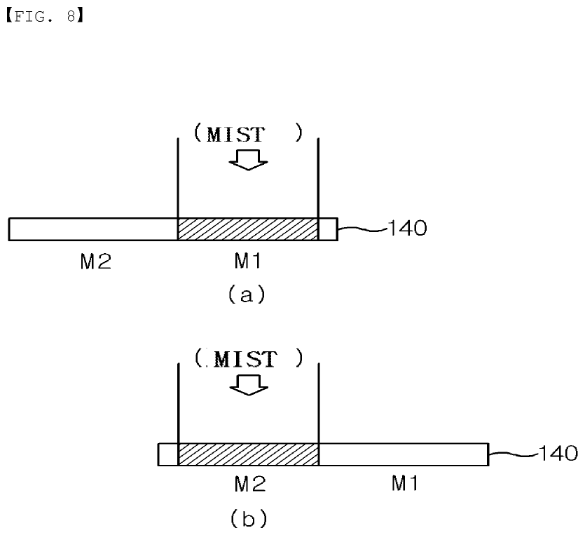

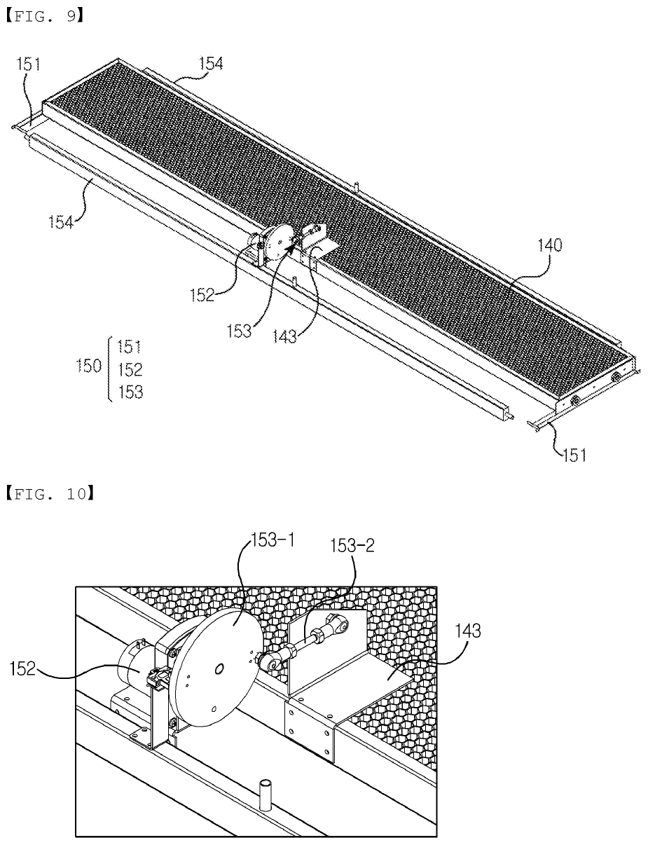

FIGS. 9 and 10 illustrate a mesh plate driving part 150 according to an exemplary embodiment, where the mesh plate driving part 150 may be formed by including a guide rail part 151 disposed at a front side and a rear side of mesh plate 140 to allow the mesh plate to move horizontally (to left/right sides), a motor part 152 and a connection part 153 configured to transmit an energy (power) to the mesh plate 140 to allow moving along the guide rail part 151 in response to rotation of the motor part 152.

The guide rail part 151 may provide a route to allow the mesh plate 140 to move horizontally while being disposed at a front side and a rear side of the mesh plate 140.

Although the figures have illustrated an example of a plate shaped rail configured to slip by allowing a distal end portion of the front side and the rear side of the mesh plate 140 to lie on the guide rail part 151, the guide rail part 151 may be variably formed to guide the mesh plate 140, and the present invention is not limited thereto.

The guide rail part 151 may be supported by a fixing bar of lengthwise direction, and distal ends of front side and rear side of the mesh plate 140 may be laid on the guide rail part 151.

At this time, the mesh plate driving part 150 may be installed on any one of the fixing bars 154 to push and pull the mesh plate 140. Here, the front side and rear side refer to a lengthwise direction of the mesh plate 140, and the left/right directions refer to a direction perpendicular to sidewise of both sides.

The connection part 153 may be formed by including a round rotation plate 153-1 rotating to the operation of the motor part 152, and a connection bar 153-2 fixed at one end to a marginal vicinity of the rotation plate 153-1 and fixed at the other end to a lengthwise center vicinity of the mesh plate 140 to push and pull the mesh plate 140 in response to the rotation of the rotation plate 153-1.

At this time, the connection bar 153-2 may be fixed to a connection bar support member 143 mounted at a center vicinity of mesh plate 140, and a center axis of the rotation plate 153-1 may be disposed to stand upright to be formed to a lengthwise direction of mesh plate 140. Furthermore, both distal ends of connection bar 153-2 may be flexibly fixed for smooth operation.

The length of connection bar 153-2 extending to a direction of the mesh plate 140 may be changed because one end of the connection bar 153-2 is fixed to an edge of the rotation plate 153-1.

That is, the connection bar 153-2 may push and pull the mesh plate 140 in response to the rotation of the rotation plate 153-1, and as a result, an area where the mists pass in the mesh plate 140 may be changed as shown in FIG. 8.

When the mesh plate driving part 150 moves the mesh plate 140 may be variably established lest the condensation of mists be generated in response to the operational situation of the mist spray device 100.

For example, the mesh plate driving part 150 may be so configured as to move the mesh plate 140 when a particular condition (state) is satisfied such as when a moving command is inputted from a manager, when the spray of sterilization water is implemented, when time has ripe for a preset moving period, and when mist condensation condition of mesh plate 140 exceeds a reference value. The mist condensation condition of mesh plate 140 may be ascertained through a sensor configured to detect the relevant condition.

<Adjustment of Conditions of Mist Spray>

The spray conditions of mists sprayed from the mist spray device 100 may require adjustments. For example, the mist shower device configured to perform sterilization and disinfection to quarantinable persons by spraying the mists may require the spray of mists to the quarantinable persons in an optimal state in response to various situations.

Although linearity (straightness) of mists may be reinforced to increase the mist spray efficiency, the degree of mist spreading may be weakened if only the linearity is strengthened to resultantly fail to provide a sufficient contact to a full body of a quarantinable person.

When the mists are not evenly sprayed, the quarantine effect may be decreased due to insufficient spread of sterilizing power to the full body of the quarantinable person.

Toward this end, the mist spray device 100 may be configured by including a spray condition (state) adjustment part 160 to adjust at least one or more of the directions, thickness, and degree of spreading of mists discharged from the mist discharge port (H1).

The spray condition adjustment part 160 may be variably formed.

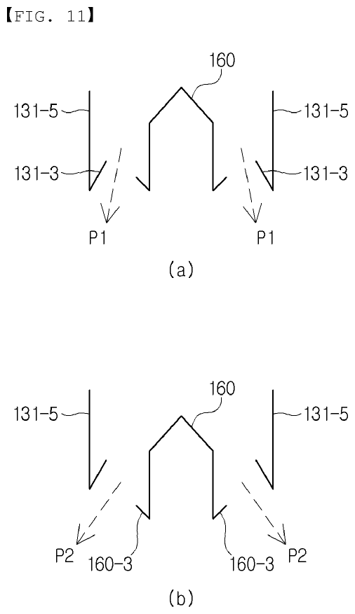

FIG. 11 explains an example in which mist spray conditions are adjusted when a spray condition adjustment part 160 ascends/descends relative to a mist discharge port (H1), where the spray condition adjustment part 160 may be formed along a center part of the mist discharge port (H1), and may ascend/descend toward an interior of the mist discharge port (H1).

Then, both lateral surfaces 131-5 forming the mist discharge port (H1) and route formed by the spray condition adjustment part 160, i.e., the shape of space flown by the mists, are determined by the current position of the spray condition adjustment part 160, whereby direction, thickness, the degree of spreading of mists discharged to outside along with air flow may be adjusted.

When the spray condition adjustment part 160 is in a position as illustrated in FIG. 11a, a mist discharge route like P1 may be formed by both lateral surfaces 131-5 forming the mist discharge port (H1) and the spray condition adjustment part 160.

When the spray condition adjustment part 160 is in a position as illustrated in FIG. 11b, a mist discharge route like P2 may be formed by both lateral surfaces 131-5 forming the mist discharge port (H1) and the spray condition adjustment part 160.

That is, the direction, the thickness, the degree of spreading of mists finally discharged to outside may be adjusted by the position of the spray condition adjustment part 160.

To form the said mist discharge route, both lateral surfaces 131-5 forming the mist discharge port (H1) and the spray condition adjustment part 160 may be formed with a variable shape.

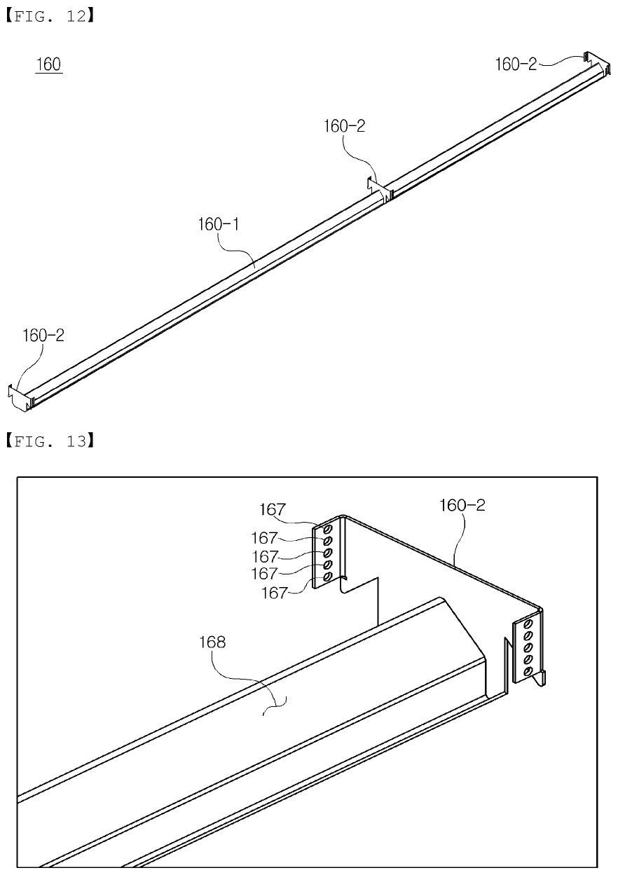

FIG. 12 illustrates the spray condition adjustment part 160 according to an exemplary embodiment of the present invention, and FIG. 13 shows an enlarged view of some portions thereof, and the spray condition adjustment part 160 may be formed by including a spray adjustment bar 160-1 lengthily formed along a center of the mist discharge port (H1), one or more installation plates 160-2 configured to attach or detach the spray adjustment bar 160-1 to or from a frame (not shown) supporting the spray condition adjustment part 160.

The spray adjustment bar 160-1 adjusts the mists discharged through the mist discharge port (H1), and therefore, relates to the shape of the mist discharge port (H1).

When the mist discharge port (H1) is formed in a narrow and lengthwise shape, the spray adjustment bar 160-1 may be also formed in the narrow and lengthwise shape along a center of the mist discharge port (H1).

The shape of the spray adjustment bar 160-1 may be variably configured to adequately adjust the spray condition of mists. In a detailed example, as illustrated in FIG. 13, the spray adjustment bar 160-1 may be formed with a shape having a slope 168 lengthwisely descending from a center toward both edges.

Then, the condition (state) of flowing mists may be adjusted while resistance to mists flowing toward the mist discharge port (H1) can be minimized.

In other words, the mists slipping out to the mist discharge port (H1) can receive a smaller resistance owing to the slope 168, and a mist discharge space may be also adjusted that is formed between both lateral surfaces 131-5 forming the mist discharge port (H1) according to the position and angle of the slope 168.

The installation plate 160-2 may enable the spray condition adjustment part 160 to be attached to or detached from the frame (not shown) of the mist spray device 100. Although three installation plates 160-2 are illustrated in the figure, the number of installation plates 160-2, the shape and the fastening structure may be variably configured, and the present invention is not limited thereto.

Each installation plate 160-2 may be formed with two or more fastening holes 167 of varying heights that enable the spray adjustment bar 160-1 to be mounted on the frame.

The figure illustrates an example in which a plurality of fastening holes 167 is lengthwisely formed on both lateral surfaces of installation plate 160-2, where the fastening holes 167 and the frame may be fastened using various fastening means such as bolts and nuts.

The spray condition adjustment part 160 may be ascended/descended depending on which number of fastening hole is to be used for fixation because fastening holes 167 are lengthwisely formed in plural number.

Each exemplary embodiment illustrated in FIGS. 12 and 13 is just one of the exemplary embodiments, and the structure for supporting and ascending/descending the spray condition adjustment part 160 may be variably configured. For example, as illustrated in the figures, although the spray condition adjustment part 160 may be formed with manual fastening structures, the structures may be automated using a motor and the like.

Referring to FIG. 11, condensate collection grooves 131-3, 160-3 may be formed at a distal end of air guide part 131 forming the mist discharge port (H1) and at a distal end of spray condition adjustment part 160 to allow waterdrops to flow down and be collected thereat when mists are condensed and waterdrops are generated as a result of condensation.

That is, even if mists are formed with microscopic particles, a portion of air guide part 131 and a portion of spray condition adjustment part 160 constantly and continuously contacted by mists may generate waterdrops due to condensation of mists.

Therefore, when the condensate collection grooves 131-3, 160-3 are formed at a distal end of air guide part 131 and at a distal end of spray condition adjustment part 160, the problematic waterdrops condensed from mists can be prevented from falling and dropping down.

There may be various methods of disposing the water flown down from the condensate collection grooves 131-3, 160-3 including, but not limited to, drying the water as it is, discharging the water through other routes and/or returning the water back into the water tank 111.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

### DESCRIPTION OF REFERENCE NUMERALS

H1: mist discharge port
30: water surface
50: raw water supply pipe
51: raw water inflow pipe
52: sterilization water outlet pipe
61: air flow
100: mist generator
111: water tank
112-1, 112-2: electrolysis modules
112-11, 112-13: electrode plate
112-12: electrode separation plate
113: mist generator
114-1: inlet
114-2: outlet
117: mist inducement means
118: porous member
121: air blower
131: air guide part
131-1: air passage
131-3: condensate collection groove
131-5: lateral surface of mist outlet
140: mesh plate
150: mesh plate driving part
151: guide rail part
152: motor part
153: connection part
153-1: rotation plate
153-2: connection bar
160: spray condition adjustment part
160-1: spray adjustment bar
160-2: installation plate
160-3: condensate collection groove
167: fastening hole
180: air inlet adjustment part
181: open/close adjustment part
181-1: open/close hole
182: main body part
182-1: air hole
200: mist shower device for sterilization and disinfection
210: upper side housing
220: left side housing
230: right side housing
251: control panel
253: transparent window

What is claimed is:

1. A mist spray device, the device comprising:
a water tank configured to contain water that is to be transformed to mists;
a mist generator configured to generate mists to above water surface of the water contained in the water tank;
an air blower configured to generate an air flow; and
an air guide part configured to form an air passage to allow the air flow generated by the air blower to be moved to a mist discharge port, wherein
the water tank is disposed with an inlet through which part of the air flows generated by the air blower is introduced and an outlet through which the mists generated by the mist generator are discharged to the air passage.

2. The mist spray device of claim 1, wherein the inlet and outlet are mutually oppositely disposed above an upper surface of the air tank, and
the mist spray device further comprises a mist inducement means to allow the air flow introduced into through the inlet to be induced to one side of the water tank,
wherein the mist inducement means is formed with a shape of a plate descending at a predetermined angle from a side formed with the outlet toward a descending side formed with the inlet.

3. The mist spray device of claim 1, further comprising an air inflow adjustment part configured to adjust an amount of air introduced into the water tank through the inlet.

4. The mist spray device of claim 1, further comprising:
one or more electrolysis modules configured to supply sterilization water to the water tank by generating the sterilization water having a sterilizing power through electrolysis; and
one or more porous members disposed inside of the water tank to allow mists heading for the outlet to pass therethrough and to drop the mists by condensing thick mist particles,
wherein the mist generator is formed by including one or more ultrasonic oscillators disposed underneath the water tank.

5. The mist spray device of claim 1,
wherein the air guide part is formed with a shape to encompass a surface formed with the inlet of the water tank, an upper surface of the water tank and a surface formed with the outlet of the water tank, and
a corner of the water tank is formed with a slope at a portion where the direction of air flow is changed, and
at least one or more air passages are so formed as to be narrowed to facilitate the air flow.

6. A mist shower device for sterilization and disinfection, the device comprising:
a housing disposed to form a passage to allow a person subject to quarantine to pass therethrough; and
the said mist spray device according to claim 1,
wherein the housing includes an upper side housing forming a ceiling, a left side housing forming a left side wall and a right-side housing forming a right-side wall,
wherein the mist spray device is disposed on an upper side housing, and
wherein the mist shower device for sterilization and disinfection is so disposed as to spray sterilizing mists downwardly from the upper side housing.

7. A mist shower device for sterilization and disinfection, the device comprising:
a housing disposed to form a passage to allow a person subject to quarantine to pass therethrough; and
the said mist spray device according to claim 2,
wherein the housing includes an upper side housing forming a ceiling, a left side housing forming a left side wall and a right-side housing forming a right-side wall,
wherein the mist spray device is disposed on an upper side housing, and
wherein the mist shower device for sterilization and disinfection is so disposed as to spray sterilizing mists downwardly from the upper side housing.
